(19) United States
(12) Patent Application Publication
(10) Pub. No.: US 2006/0066449 A1
(43) Pub. Date: Mar. 30, 2006

(54) RFMON: devices and methods for wireless monitoring of patient vital signs through medical sensor readings from passive RFID tags

(75) Inventor: Richard C. Johnson, Poquott, NY (US)

Correspondence Address: YOUNG LAW FIRM;A PROFESSIONAL CORPORATION, 4370 ALPINE ROAD SUITE 106, PORTOLA VALLEY, CA, 94028, US

(73) Assignee: Industrial Widget Works Company, Poquott, NY (US)

(21) Appl. No.: 11/222,241

(22) Filed: Sep. 7, 2005

Related U.S. Application Data
(60) Provisional application No. 60/608,279, filed on Sep. 8, 2004.

(51) Int. Cl. G08B 1/08; A61B 5/00

(52) U.S. Cl. 340/539.12; 600/300

(57) ABSTRACT

A system for monitoring a selected physiological activity of a person includes a passive wireless tag including a sensor for monitoring the selected physiological activity of the person and a Radio Frequency Identification (RFID) tag coupled to the sensor. The RFID tag may include a unique identifier and may be configured to, when polled, generate, store and transmit a monitoring signal representative of the selected physiological activity and of the RFID tag unique identifier. A monitoring and processing station may be configured to poll the passive wireless tag and to receive and process the monitoring signal transmitted from the passive wireless tag.

This application claims the benefit under 35 U.S.C. §119(e), of U.S. provisional application Ser. No. 60/608,279, filed Sep. 8, 2004, which application is hereby incorporated herein by reference in its entirety.

### BACKGROUND OF THE INVENTION

1. Field of the Invention

The present inventions relate to the field of wireless patient monitoring of patient vital signs and other signals from RFID patient tags, which include medical monitoring sensors on the patient.

2. Description of the Related Art

Conventionally, sensors such as used in hospital setting for monitoring patient vital signs inferred from tiny currents in the patient's skin are attached to wires that are further connected to monitor processing stations. The monitor processing stations carry out analog to digital processing, storage, analysis, display, and control functions. These sensors and their trailing wires severely constrain patient mobility and can be uncomfortable and awkward, particularly for those on bed rest for extended periods of time. The sheer number and weight of the many sensor wires attached to their body may leave patients feeling like Gulliver did when he was roped down by the Lilliputians. The wires can become entangled and require a significant effort to untangle them, often requiring a nurse to disconnect them from the monitor processing station. Aid from a nurse or other attendant is also required for a patient to get out of bed and visit the bathroom or even to exercise by walking the hospital halls. Moreover, each time that the wires become detached for whatever reason, the patient monitor generates an alarm noise and an attendant must come to the patient's bedside and reattach the wire leads. These alarms annoy all patients within earshot, distract the nurses and attendants, and in general constitute both a false alarm and a frequently re-occurring chore to re-attach the wires. From a patient care point of view, it may also be appreciated that the patient's vital signs are no longer being monitored when the patient, whether intentionally or unintentionally, has disconnected the wires from the sensors attached to their body. This means that patient are typically not monitored when they are in the bathroom, moving about in their room, being conveyed by gurney to imaging or other medical services within the hospital, or walking the hallways for exercise. Current adaptations of wireless technologies in patient monitors carry the wireless signal from the monitor to the processing station or the nurses' station. Other adaptations allow a mobile patient to be wired to a portable wireless monitor/transmitter. However, the patient is still wired from the adhesive sensors attached to his or her body to the monitor processing station or portable monitor. Patient smocks have few pockets able to accommodate the portable monitor and such a device as a Holter monitor usually records signals and resend them through a telephone. This implementation has some advantages for the transmission of signals from the individual patient monitors forward to nurses' stations, but provides no relief to the problem cited above as the patient is still tethered by wire to the bedside or mobile monitor processing station. Furthermore, the mobile monitors frequently lack many features of the conventional monitor and are relatively expensive.

From the foregoing, it may be appreciated that the ability to monitor patients wirelessly from patient to monitor would benefit both patients and hospital care providers. Such monitoring ability would also greatly benefit others. For example, those having dangerous jobs, such as policemen, firemen, soldiers entering combat, medics, rescuers and other first responders would benefit from wireless monitoring of vital signs. Additionally, transportation personnel such as pilots or others with critically important job functions would also benefit from wireless monitoring. Indeed, such benefit extends not only to the pilot, but also to any passengers the pilot may be transporting. Ideally, such means for wirelessly monitoring vital signs would be comfortable to wear, secure, easy to implement, and be sufficiently inexpensive as to justify the cost of replacing the wired sensors and associated monitoring equipment.

### BRIEF DESCRIPTION OF THE DRAWINGS

FIG. 1 illustrates aspects of a system for wireless patient monitoring of patient's vital signs, according to an embodiment of the present invention.

FIG. 2 shows a wireless patient monitor, according to an embodiment of the present invention.

FIG. 3 is a flowchart illustrating an embodiment of a method for wireless patient monitoring of patient vital signs, according to an embodiment of the present invention.

### SUMMARY OF THE INVENTION

According to an embodiment thereof, the present invention may be seen as a system for monitoring a selected physiological activity of a person. The system may include a passive wireless tag including a sensor for monitoring the selected physiological activity of the person and a Radio Frequency Identification (RFID) tag coupled to the sensor, the RFID tag including a unique identifier, the passive wireless tag being configured to, when polled, generate, store and transmit a monitoring signal representative of the selected physiological activity and of the RFID tag unique identifier, and a monitoring and processing station configured to poll the passive wireless tag and to receive and process the monitoring signal transmitted from the passive wireless tag.

The passive wireless tag may further include an analog to digital (A/D) converter coupled to the sensor, a memory buffer coupled to the A/D converter and an antenna coupled to the memory buffer, the antenna being configured to at least transmit the monitoring signal to the monitoring and processing station. The RFID tag may be configured to store the RFID unique identifier in the memory buffer. The monitoring signal may include the stored unique RFID unique identifier. The passive wireless tag may be configured for single use and may be disposable. The RFID tag may be configured to transmit the monitoring signal only when polled by the monitoring and processing station. The passive wireless tag further may include a battery whose power has run too low to power the passive wireless tag. The passive wireless tag may be configured to transmit the monitoring signal when polled by the monitoring and processing station when battery power decreases below a predetermined threshold. The system may also include a uniquely identified access point that is configured to receive the monitoring signal and to transmit the received monitoring signal to the monitoring and processing station. The access point may be coupled to the monitoring and processing station over a communication network. A digital value representative of the monitoring signal may be periodically written to the memory buffer, each subsequent writing of the digital value overwriting a previously written digital value. The memory buffer may be configured as a FIFO and a digital value representative of the monitoring signal may be periodically written to the memory buffer. The passive wireless tag may be further configured to write the monitoring signal to the memory buffer more frequently than the monitoring and processing station receives monitoring signals. The monitoring and processing station may store a location of the uniquely identified access point, and a location of the passive wireless tag may be inferred the stored location of the access point that transmitted the monitoring signal. The monitoring signal may be encrypted.

According to another embodiment thereof, the present invention may also be seen as a system for monitoring physiological activity of a person that may include a plurality of passive wireless tags, each including a sensor for monitoring the physiological activity of the person and a Radio Frequency Identification (RFID) tag coupled to the sensor, the RFID tag including a unique identifier, the passive wireless tags being configured to, when polled, generate, store and transmit a monitoring signal representative of the selected physiological activity; a plurality of uniquely identified access points configured to poll the plurality of passive wireless tags and to receive and to re-transmit the monitoring signal, the plurality of access points being geographically distributed within a predetermined area, and a monitoring and processing station configured to control the plurality of uniquely identified access points and to receive and process the monitoring signal re-transmitted from the uniquely identified access points.

One or more of the plurality of access points may be configured to poll passive wireless tags that are within a predetermined range and, responsive thereto, to receive monitoring signals from passive wireless tags within the predetermined range. Each of the plurality of access points may be coupled to the monitoring and processing station by a communication network. The passive wireless tag may further include a battery whose power has run low or out.

According to yet another embodiment, the present invention is a method of monitoring physiological activity of a person. The method may include steps of providing a passive wireless tag including a unique identifier, a sensor for monitoring the selected physiological activity of the person and a Radio Frequency Identification (RFID) tag coupled to the sensor, the RFID tag including a unique identifier, the passive wireless tag being configured to, when polled, generate, store and transmit a monitoring signal representative of the selected physiological activity and of the unique identifier; providing a plurality of uniquely identified access points configured to receive and to re-transmit the monitoring signal, the plurality of access points being geographically distributed at known locations within a predetermined area; providing a monitoring and processing station configured to control the plurality of uniquely identified access points over a communication network; adhering the passive wireless tag to the person; generating a polling signal to poll the passive wireless tags to cause the passive wireless tags within range of the polling signal to generate, store and transmit the monitoring signal; receiving the transmitted monitoring signal by one or more of the plurality of uniquely identified access points and re-transmitting the received monitoring signal over the communication network, and receiving the re-transmitted monitoring signal in the monitoring and processing station.

The first providing step may be carried out with the passive wireless tag further including an analog to digital (A/D) converter coupled to the sensor, a memory buffer coupled to the A/D converter and an antenna coupled to the memory buffer, the antenna being configured to at least transmit the monitoring signal to the monitoring and processing station. The RFID tag may be configured to store the unique identifier in the memory buffer and wherein the monitoring signal in the generating and receiving steps is configured to include the unique identifier. The first providing step may be carried out with passive wireless tag being configured for single use and disposability. The first providing step may be carried out with the passive wireless tag further including a battery whose power has run low or out. The first providing step may be carried out with the passive wireless tag being configured to transmit a signal indicative of low battery power. The first providing step may be carried out with the passive wireless tag being further configured to transmit the monitoring signal when polled by the monitoring and processing station when battery power decreases below a predetermined threshold. A step of periodically writing a digital value representative of the monitoring signal to the memory buffer may also be carried out, each subsequent writing of the digital value overwriting a previously written digital value. The memory buffer may be configured as a FIFO and the method may further include a step of periodically writing a digital value representative of the monitoring signal to the memory buffer. The method may also include a step of writing the monitoring signal to the memory buffer more frequently than the monitoring and processing station receives monitoring signals. The monitoring and processing station may carry out a step of storing the known locations of the plurality of access points, and wherein the method further includes a step of inferring a location of each passive wireless tag from which a monitoring signal is received.

### DESCRIPTION OF THE PREFERRED EMBODIMENTS

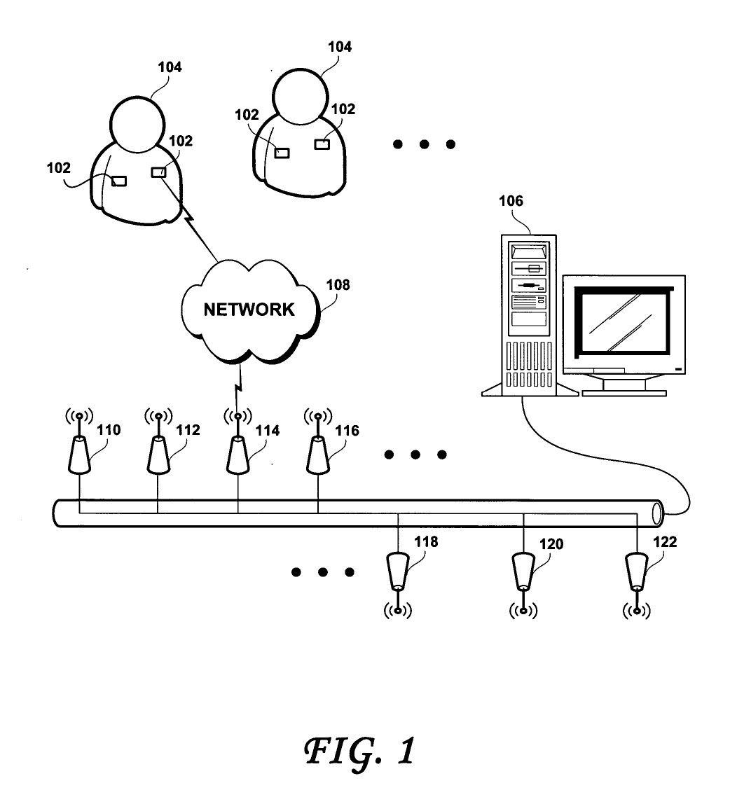

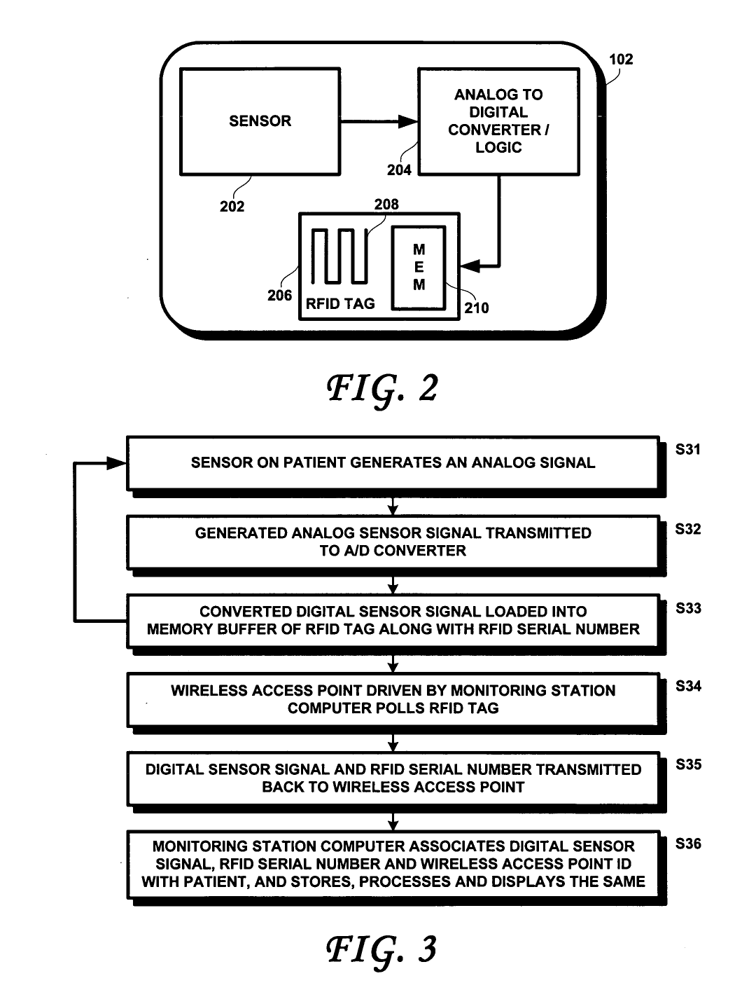

Considering now FIGS. 1, 2 and 3 collectively, embodiments of the present invention may utilize Radio Frequency Identification (hereafter, RFID) type emitters such as shown at 206 in FIG. 2 to transfer information from a wireless patient tag 102 attached to a human or animal body 104, without wires, to a monitoring and processing station 106. The monitoring and processing station 106 may be portable or may be in a fixed location and may then receive the signal containing the information generated by the wireless patient tag 102 and process it, store it, display it, and relay it as appropriate.

A wireless patient tag 102, according to an embodiment of the present invention, may include a sensor 202, an analog to digital converter and associated logic (hereafter, A/D converter) 204 coupled to the sensor 202 and an RFID tag 206, which includes an antenna 208 and an on-board memory buffer 210. As shown at step S3 1, in use, the wireless patient tag 102 is attached to the patient 104 and the sensor 202 therein generates an analog signal that is characteristic of a vital sign or other physiological activity to be measured. The analog signal generated by the sensor 202 may then be transmitted to the A/D converter 204, as shown at S32. The A/D converter may then convert the received analog signal into a digital bit string result that is then loaded by a logical circuit into the (e.g., passive) memory buffer 210 of the RFID tag 206, as indicated at S33. Each of the RFID tags 210 may have a unique RFID serial number already stored in the memory buffer. Therefore, in use, each of the memory buffers 210 of each RFID tag 206 of each wireless patient tag 102 will have its own unique RFID serial number associated therewith. This unique RFID serial number, together with the converted digital result also stored in the memory buffer 210, may then be periodically transmitted back to the monitoring and processing station(s) 106.

According to one embodiment of the present invention, all or a selected one or ones of the wireless RFID access points 110 may be driven by the monitoring and processing station 106 (or by some other suitable device) to poll nearby wireless patient tags 102 and receive back the contents of their respective memory buffers 210. This step is also shown at S34 in FIG. 3. The RFID tag 206 of the wireless patient tag 102 may be passive, in that it may utilize the energy received on the send/receive antenna 208 to (1) allow the sensor-detected electrical signal to be sent to the A/D converter 204, 2) pass through the logic circuit, which will 3) load it into the correct location in the memory buffer 210, and 4) send the contents of the memory buffer 210 (both the serial ID of the RFID tag and the A/D converted sensor reading as a single string (for example) in a signal out over the antenna 208 to be received by the wireless access points 110-120. The wireless patient tags 102 may be fabricated and may be configured to be used once and thereafter discarded. Moreover, the wireless patient tags may be configured to be rendered inoperative if removed from the patient. Other embodiments of the present invention envisage that the wireless patient tags 102 are configured to send the contents of their respective memory buffers 210 asynchronously to the monitoring and processing station or stations 106, without having to be polled thereby. These tags 102 may be configured to include (1) a battery or current source and (2) wiring logic which controls the emission of the combined RFID (e.g., sensor digital+serial number) signal to whatever receivers may be in the area. The range of the present wireless patient tags may be greatly increased through the use of such batteries or other local source of energy, as compared to the passive configuration.

Indeed, according to another embodiment of the present invention, the present RFMon™ wireless patient tags may be powered by an internal power source, such as one or more small batteries. In that case, the wireless patient tag may have greater range and may send its signal asynchronously; that is, without being polled by the wireless access points 110-120. Such wireless patient tags may be characterized as active, as opposed to wireless patient tags devoid of such internal power source, which may be characterized as passive (responding only when polled). A battery powered wireless patient tag, according to an embodiment of the present invention, may be configured to store sufficient energy to announce its low battery status as a last resort. Alternatively, a static memory state may be altered on the RFID tag 206 when the battery is low and the RFID 206 of the wireless patient tag 102 may become passive (i.e., able to respond only when polled by the wireless access points 110-120) absent power, and may be configured to reflect its status as “Present but Battery Impaired” when polled. The batteries may be recharged through induction (for example) to maintain active or semi active tags. Induction charging of the batteries may be carried out at the patient's bedside or at other suitable location such as out in the field.

As shown at S35, the digital packet generated by the wireless patient tag 102 (including, for example, at least the serial number of the RFID tag 206 plus the current sensor digital signal), responsive to the polling from the monitoring and processing station 106, may be sent over the network 108 (e.g., a WiFi network according to the IEEE 802.11 standard, for example, or an Ethernet network) to one or more of the plurality of uniquely identified wireless access points 110-120 within range of the RFID tag 206. The transmitted and received digital packet may then be transmitted to the monitoring and processing station(s) 106. According to an embodiment of the invention, as shown at S36, the monitoring and processing station 106 may then form an association between the uniquely identified wireless access points 110-120, the digital packet (including, for example, the including at least the serial number of the RFID tag 206 plus the current sensor digital signal) and the ID of the uniquely identified wireless access points 110-120 having received the digital packet and the patient. Other metrics may also be associated with the ID of the wireless access point 110-120 and the digital packet such as, for example, patient name, other patient identifier(s), the location of the patient, the function of the wireless patient tag 102 to thereby build a series of readings that may be interpreted as a measure of a patient vital sign and medical condition over time as well as by location. The information received by the monitoring and processing station 106 may be then be stored, processed and displayed as required. These features are invaluable for first responders and military personnel, who may easily prepare for dangerous duty by wearing tags 102. Then, if the tag 102 wearing person were injured, a medic or emergency personnel would be able to immediately assess their medical condition. Indeed, in any dangerous job, whether first responder policemen or firemen or soldiers entering combat, wireless sensors such as described herein will allow medics, rescuers, and other authorities to (1) locate, (2) identify, and (3) assess the medical condition of individuals. A monitor brought into proximity to the wireless sensors will, according to one embodiment of the present invention, poll the sensors and convey the wearer's vital signs immediately to the medic or others. Depending upon what is measured, it is even possible that the pattern of vital signs, like fingerprints, will identify an individual uniquely.

As shown by the arrow from S33 to S31, the sensor 202 within the wireless patient tag 102 may continually generate an analog signal of the measured physiological activity. This analog signal may be converted to a digital string and stored in the buffer memory 210 of the RFID tag 206 of the wireless patient tag 102. This sequence occurs continuously and the digital string generated by the A/D converter 204 may be written to the buffer memory 210 in such a manner, according to an embodiment of the present invention, that is overwrites the previous value stored therein. In this manner, only the most recent digital string is stored in the buffer memory 210, thereby insuring that only the digital packet containing the most up-to-date data is transmitted to the wireless access point 110-120. According to other embodiments, the buffer memory 210 may be organized as a First In, First Out (FIFO) buffer, in which case many digital packets may be pushed into the buffer by the A/D converter 204 and popped out of the buffer memory when the wireless access points 110-120 poll the wireless patient tag 102. The wireless access points 110-120 may be controlled by the monitoring and processing station 106 to poll the wireless patient tags 102 at a predetermined schedule, which may be slower than the rate at which new digital bit strings are stored in the memory buffer 210 of the RFID tag 206. Therefore, not every digital packet may (or need) be transmitted back to the monitoring and processing station 106 through the wireless access points 110-120. That is, the data in the memory buffer 210 may be replaced much more often than the digital packets (including at least the serial number of the RFID tag 206 and the digital bit string stored in the memory buffer 210 by the A/D converter 204) may be transmitted back to the monitoring and processing station 106.

The patient's location as of last receipt of information from the patient's wireless patient tag 102, may be derived from the ID of the wireless access point 110-120 having received the digital packet. Indeed, each wireless access point 110-120 may be associated with its physical location in the hospital or clinic (for example). By identifying which wireless access point received the digital packet, the patient's current location may be inferred. For example, wireless access point 112 may be over the patient's bed, whereas access point 114 may be in the patient's restroom and wireless access point 116 may be in the hallway outside the patient's room. In this manner, the patient's vital signs and current location may be continuously tracked over time, without requiring the patient to be tethered to a monitoring station while in bed or unmonitored while ambulatory.

Conventional wireless medical monitors may be wireless from the monitoring station or portable processing box back to a console at the nurses' station. Embodiments of the present invention are wireless between the patient and the monitoring and processing station 106, which then itself may be wirelessly connected to the nurse's station or to a central station or may be hard-wired from its location in halls or rooms.

Embodiments of the present invention may transmit a digital packet from the wireless patient tags, to the wireless access point 110-120 (e.g., a radio frequency receiving antenna connected to networks such as WiFi or Ethernet) to the monitoring and processing station 106, leaving a patient free to move about, visit the bathroom, walk the halls, all the while constantly transmitting vital signs and other data to the polling wireless access points 110-120 staged in rooms and halls where a patient might move. Individual patients may be equipped with identified sets of tagged wireless patient tags 102, such that the wireless patient tags of multiple patients could communicate their digital packets to a single monitoring and processing station 106. Because each digital packet includes the unique ID of the RFID tag 206 and because each wireless patient tag is uniquely identified and associated with an identified patient, the sensor information form the sensor 202 of the wireless patient tag is associated only with the appropriate individual, in a secure manner. According to other embodiments, each digital packet may be encrypted, and the encryption key needed to decrypt and read the information contained in the digital packet may be different for each patient.

The wireless patient tag may include a small metal receptacle or female coupling for the RFID tag 206 (which tag is commercially available) and the A/D converter 204, which small metal receptacle may be made to accommodate the metal nipple or short male nubbin on an standard adhesive sensor for, for example, an electrocardiogram machine or for a vital signs monitor. Alternatively, the tag 102 may be layered into the sensor patch itself. The benefits conferred by the disclosed embodiments of the present invention are many, including:
increasing patient safety by allowing constant monitoring in spite of patient movement, whether moving to the bathroom from the bed or whether being taken to another floor for medical procedures;
insuring that the patient's vital signs remain monitored without specific wiring between the patient and the collecting instrument(s);
allowing data collection instruments such as the monitoring and processing station 106 to receive communicated digital packets that may include at least the following two elements: a serial number (bit string) for the RFID tag 206 and, concatenated to it, a bit string which contains a digital representation of the patient's vital sign function, which originated in an analog reading by a sensor 202 and was processed by an A/D converter 204 and a digital logic circuit (standard or custom ICs) into that digital representation and conveyed through wireless access points 110-120 to a central location (the monitoring and processing station 106, for example), where the patient information and pre-registered RFID tag serial numbers may allow the data to be interpreted, associated with the patient, with the current time and date and the patient's location, by both the medical and billing systems. This last function is important in that other systems rely on patient identification at the bedside rather than later association with pre-registered RFID tags. To insure the integrity of the system and continued safety of the wearer, the wireless patient tags 102 and their contained RFID tags 206 are preferably scanned at the monitoring and processing station 106 or some other central location before being taken to the patient, and patient-specific ID and date/time should be printed on adhesive tags and attached to each wireless patient tag 102 and should preferably be individually numbered. The nurse or technician may then check these patient-specific IDs printed on the adhesive tags on the wireless patient tags 102 against the patient's wrist band and apply only the correct and known wireless patient tags 102 to the patient. Other security measures may be taken in addition or in place of the aforementioned security measures, as those of skill in this art may appreciate.

Note that other alternatives, such as identifying the patient at bedside and entering the patient-specific ID of each wireless patient tag 102 into a monitoring and processing station 106 at that point poses additional risks of data entry errors, especially as patients are moved from one bed to another, which runs the risk of repeating earlier errors of patient identification via charts and bed locations.

The monitoring of vital signs in patients during physical therapy treatment, the monitoring of vital signs in healthy persons performing exercise, and the use of such wireless patient monitors for emergency medical monitoring of the victims of accident or sudden illness are examples of the wide applications of the embodiments of the present invention described herein. Other medical devices based on a plurality of sensors (such as electrocardiograms, for example) may make advantageous use of the wireless patient tags disclosed herein. The present wireless patient tags may be applied to even uncooperative or convulsing patients with greater ease than conventional patient sensors, which require wires to communicate patient data to a monitor or communications device.

One advantage of the present wireless patient tags 102 is that they are largely unaffected by patient thrashing or short range movement. Constant monitoring insures that a patient in a hospital bathroom does not leave a monitored situation; a patient having an ischemic or other cardiac reaction in a bathroom would not have to rely on his or her (perhaps rapidly waning) ability to pull a red cord or to push a button to alert a nurse. Rather, the patient's vital signs are continuously monitored, even when the patient is ambulatory, moving about, or closeted in the bathroom.

The cost advantage to providers of medical care is as large as the increase in the above-described effective monitoring of patients. Nurse and attendant staffing are often strained by the need to constantly tend to the wired patient sensors, in that they must often untangle the wires even to roll a patient over on his or her side, unhook them to let them out of bed, and re-connect them every time the patient is moved, visits the bathroom or exercises. Freeing nurses and other healthcare workers from the need to tend to the wired monitors frees them to care for their patients, rather than their patient's technology.

By disposing wireless access points 110-120 throughout the hospital or other healthcare setting allows the patient to be transported through the hospital for testing or other procedures while maintaining a constant monitoring of the patient's vital signs or other measured physiological activity. For example, the wireless access points may allow continuous monitoring of patients in a wide variety of hospital situations, such as x-ray or other testing, measurement, or scanning, which currently escape the conventional wired monitoring system.

In addition to increasing the time during which patients are monitored, patient comfort is greatly enhanced. Patients who can visit the bathroom without waiting for a nurse or attendant are happier patients. Exercise available by walking the halls is more accessible and more likely with wireless patient tags; ambulatory patients are also far less likely to escape monitoring with the use of the embodiments of the present invention disclosed herein.

In fact, the wireless patient sensor tags 102 described herein may be affixed prior to need as described above for firemen and soldiers and pilots. The tags 102 may be affixed to any individual entering a situation, whether exercise or danger, where monitoring is indicated and the polling stations available. In cases where polling stations are unavailable, the polling stations themselves may be configured to be rugged and portable so as to enable them to be brought into the proximity of the tags 102 only in the case of an emergency situation.

The present invention has been described in connection with the preferred embodiments; however, it is understood that many alternatives are possible without departing from the scope of the invention. It is also understood that individuals not generally regarded as patients, e.g. persons undergoing an exercise program, other fitness activity or routine physiological or sports-related monitoring, or anyone going into potential danger may benefit from further embodiments of this invention.

What is claimed is:

1. A system for monitoring a selected physiological activity of a person, comprising:
a passive wireless tag including a sensor for monitoring the selected physiological activity of the person and a Radio Frequency Identification (RFID) tag coupled to the sensor, the RFID tag including a unique identifier, the passive wireless tag being configured to, when polled, generate, store and transmit a monitoring signal representative of the selected physiological activity and of the RFID tag unique identifier;
a monitoring and processing station configured to poll the passive wireless tag and to receive and process the monitoring signal transmitted from the passive wireless tag.

2. The system for monitoring a selected physiological activity of claim 1, wherein the passive wireless tag further includes an analog to digital (A/D) converter coupled to the sensor, a memory buffer coupled to the A/D converter and an antenna coupled to the memory buffer, the antenna being configured to at least transmit the monitoring signal to the monitoring and processing station.

3. The system for monitoring a selected physiological activity of claim 2, wherein the RFID tag is configured to store the RFID unique identifier in the memory buffer.

4. The system for monitoring a selected physiological activity of claim 3, wherein the monitoring signal includes the stored unique RFID unique identifier.

5. The system for monitoring a selected physiological activity of claim 1, wherein the passive wireless tag is configured for single use and is disposable.

6. The system for monitoring a selected physiological activity of claim 1, wherein the RFID tag is configured to transmit the monitoring signal only when polled by the monitoring and processing station.

7. The system for monitoring a selected physiological activity of claim 1, wherein the passive wireless tag further includes a battery whose power has run too low to power the passive wireless tag.

8. The system for monitoring a selected physiological activity of claim 7, wherein the passive wireless tag is configured to transmit the monitoring signal when polled by the monitoring and processing station when battery power decreases below a predetermined threshold.

9. The system for monitoring a selected physiological activity of claim 1, further comprising a uniquely identified access point that is configured to receive the monitoring signal and to transmit the received monitoring signal to the monitoring and processing station.

10. The system for monitoring a selected physiological activity of claim 9, wherein the access point is coupled to the monitoring and processing station over a communication network.

11. The system for monitoring a selected physiological activity of claim 2, wherein a digital value representative of the monitoring signal is periodically written to the memory buffer, each subsequent writing of the digital value overwriting a previously written digital value.

12. The system for monitoring a selected physiological activity of claim 2, wherein the memory buffer is configured as a FIFO and wherein a digital value representative of the monitoring signal is periodically written to the memory buffer.

13. The system for monitoring a selected physiological activity of claim 2, wherein the passive wireless tag is further configured to write the monitoring signal to the memory buffer more frequently than the monitoring and processing station receives monitoring signals.

14. The system for monitoring a selected physiological activity of claim 9, wherein the monitoring and processing station stores a location of the uniquely identified access point, and wherein a location of the passive wireless tag may be inferred the stored location of the access point that transmitted the monitoring signal.

15. The system for monitoring a selected physiological activity of claim 1, wherein the monitoring signal is encrypted.

16. A system for monitoring physiological activity of a person, comprising:
a plurality of passive wireless tags, each including a sensor for monitoring the physiological activity of the person and a Radio Frequency Identification (RFID) tag coupled to the sensor, the RFID tag including a unique identifier, the passive wireless tags being configured to, when polled, generate, store and transmit a monitoring signal representative of the selected physiological activity;
a plurality of uniquely identified access points configured to poll the plurality of passive wireless tags and to receive and to re-transmit the monitoring signal, the plurality of access points being geographically distributed within a predetermined area;
a monitoring and processing station configured to control the plurality of uniquely identified access points and to receive and process the monitoring signal re-transmitted from the uniquely identified access points.

17. The system for monitoring physiological activity of claim 16, wherein at least one of the plurality of access points is configured to poll passive wireless tags that are within a predetermined range and, responsive thereto, to receive monitoring signals from passive wireless tags within the predetermined range.

18. The system for monitoring physiological activity of claim 16, wherein each of the plurality of access points is coupled to the monitoring and processing station by a communication network.

19. The system for monitoring physiological activity of claim 16, wherein the passive wireless tag further includes a battery whose power has run out.

20. A method of monitoring physiological activity of a person, comprising the steps of:
providing a passive wireless tag including a unique identifier, a sensor for monitoring the selected physiological activity of the person and a Radio Frequency Identification (RFID) tag coupled to the sensor, the RFID tag including a unique identifier, the passive wireless tag being configured to, when polled, generate, store and transmit a monitoring signal representative of the selected physiological activity and of the unique identifier;
providing a plurality of uniquely identified access points configured to receive and to re-transmit the monitoring signal, the plurality of access points being geographically distributed at known locations within a predetermined area;
providing a monitoring and processing station configured to control the plurality of uniquely identified access points over a communication network;
adhering the passive wireless tag to the person;
generating a polling signal to poll the passive wireless tags to cause the passive wireless tags within range of the polling signal to generate, store and transmit the monitoring signal;
receiving the transmitted monitoring signal by one or more of the plurality of uniquely identified access points and re-transmitting the received monitoring signal over the communication network, and
receiving the re-transmitted monitoring signal in the monitoring and processing station.

21. The method of claim 20, wherein the first providing step is carried out with the passive wireless tag further including an analog to digital (A/D) converter coupled to the sensor, a memory buffer coupled to the A/D converter and an antenna coupled to the memory buffer, the antenna being configured to at least transmit the monitoring signal to the monitoring and processing station.

22. The method of claim 20, wherein the RFID tag is configured to store the unique identifier in the memory buffer and wherein the monitoring signal in the generating and receiving steps is configured to include the unique identifier.

23. The method of claim 20, wherein the first providing step is carried out with passive wireless tag being configured for single use and disposability.

24. The method of claim 20, wherein the first providing step is carried out with the passive wireless tag further including a battery whose power has run low.

25. The method of claim 23, wherein the first providing step is carried out with the passive wireless tag being configured to transmit a signal indicative of low battery power.

26. The method of claim 23, wherein the first providing step is carried out with the passive wireless tag being further configured to transmit the monitoring signal when polled by the monitoring and processing station when battery power decreases below a predetermined threshold.

27. The method of claim 21, further comprising a step of periodically writing a digital value representative of the monitoring signal to the memory buffer, each subsequent writing of the digital value overwriting a previously written digital value.

28. The method of claim 21, wherein the memory buffer is configured as a FIFO and wherein the method further includes a step of periodically writing a digital value representative of the monitoring signal to the memory buffer.

29. The method of claim 21, further comprising the step of writing the monitoring signal to the memory buffer more frequently than the monitoring and processing station receives monitoring signals.

30. The method of claim 20, wherein the monitoring and processing station carries out a step of storing the known locations of the plurality of access points, and wherein the method further includes a step of inferring a location of each passive wireless tag from which a monitoring signal is received.
